SEQUENCE LISTING (4 sequences)

SEQ ID NO: 1 - protein, 5 aa, Artificial Sequence
  Description of Artificial Sequence Synthetic peptide
YIGSR

SEQ ID NO: 2 - protein, 5 aa, Artificial Sequence
  Description of Artificial Sequence Synthetic peptide
IKVAV

SEQ ID NO: 3 - protein, 47 aa, Artificial Sequence
  Description of Artificial Sequence Synthetic polypeptide
GGYGGGPCGPPGPPGPPGPPGPPGFXGERGPPGPPGPPGPPGPPGPC

SEQ ID NO: 4 - protein, 9 aa, Artificial Sequence
  Description of Artificial Sequence Synthetic peptide
GGGGRGDSP

(19) United States
(12) Patent Application Publication
(10) Pub. No.: US 2016/0243281 A1
(43) Pub. Date: Aug. 25, 2016

(54) HYALURONIC ACID AND ALGINATE HYDROGEL COMPOSITION

(71) Applicant: THE REGENTS OF THE UNIVERSITY OF CALIFORNIA, Oakland, CA (US)

(72) Inventor: Jonathan Kent LEACH, Davis, CA (US)

(21) Appl. No.: 15/027,236

(22) PCT Filed: Oct. 1, 2014
(86) PCT No.: PCT/US14/58553

§ 371 Date: Apr. 4, 2016

Related U.S. Application Data
(60) Provisional application No. 61/887,199, filed on Oct. 4, 2013.

(51) Int. Cl. A61L 27/26; A61L 27/12; A61L 27/22; A61L 27/50; A61L 27/52; A61L 27/20

(52) U.S. Cl. CPC A61L 27/26; A61L 27/52; A61L 27/20; A61L 27/227; A61L 27/50; A61L 27/12; A61L 2300/412; A61L 2300/25; A61L 2300/606; A61L 2430/02

(57) ABSTRACT

A hydrogel composition comprising an alginate polymer and a hyaluronic acid polymer is a useful polymer scaffold for mammalian tissue engineering. To enable cell adhesion, adhesion peptides may be covalently linked to a portion of the alginate and/or hyaluronic acid. This alginate-hyaluronic acid-adhesion peptide hydrogel composition localizes cells and other biological constituents to promote tissue repair at a site of tissue defect, while controlling the speed of gelation and resorption time.

### CROSS-REFERENCE TO RELATED APPLICATIONS

This application claims priority to U.S. Provisional Application No. 61/887,199, filed on Oct. 4, 2013, which is incorporated by reference herein in its entirety.

### BACKGROUND

1. Technical Field

This disclosure relates to hydrogel compositions useful for tissue engineering, and more specifically, to the use of a hydrogel composition composed of alginate, hyaluronic acid, and cell-instructive peptides, which is useful for tissue repair.

2. Description of the Related Art

The natural formation and repair of mammalian tissues occurs via complex intra- and extracellular events. Tissue engineering uses a combination of synthetic and natural materials and bioactive moieties to produce engineered tissue, which can repair or replace diseased or damaged mammalian tissue. Polymer scaffolds are biomaterials used in tissue engineering that may stimulate cellular processes and cell adhesion, which in turn promote cell survival and proliferation. Different polymer scaffolds have different structural and biological properties, and thus polymer scaffolds can be engineered for specific applications. It is desirable to produce polymer scaffold hydrogels for use in mammalian tissue engineering. Specifically, it is desirable to produce a polymer scaffold hydrogel that localizes cells and other biological constituents, and promotes tissue repair at a site of tissue defect in a mammal, while controlling the speed of gelation and resorption time.

### BRIEF DESCRIPTIONS OF THE DRAWINGS

The disclosed embodiments have other advantages and features which will be more readily apparent from the following detailed description of the invention and the appended claims, when taken in conjunction with the accompanying drawings, in which:

Figure (or “Fig.”) 1 illustrates the porous morphology of unmodified and RGD-coupled acellular alginate gels, according to an embodiment.

FIG. 2 illustrates the porous morphology of unmodified and RGD-coupled cellularized alginate gels, according to an embodiment.

FIG. 3 illustrates the gross morphology of an RGD-modified alginate gel and mineralized microspheres after gelation, according to an embodiment.

FIG. 4 illustrates morphologies of encapsulated ovine MSCs in multiple gels, according to an embodiment.

FIG. 5 illustrates ovine MSCs entrapped within hydrogels and examined after 3 weeks of culture in vitro via histology, according to an embodiment.

FIG. 6 illustrates in vitro data analyzing early (alkaline phosphatase) and late (calcium) osteogenic differentiation of ovine MSCs when entrapped in alginate hydrogels and cultured in vitro, according to an embodiment.

FIG. 7 illustrates an RGD-modified hydrogel repair process for bilateral iliac defects in sheep, according to an embodiment.

FIG. 8 illustrates radiographs of pelvic portions of a sheep control group treated with acellular alginate hydrogels.

FIG. 9 illustrates in vivo micro-computed tomography images of pelvic portions of a sheep control group treated with acellular alginate hydrogels.

FIG. 10 illustrates 3-dimensional in vivo micro-computed tomography images of pelvic portions of a sheep control group treated with acellular alginate hydrogels.

FIG. 11 illustrates radiographs of pelvic portions of sheep 12 weeks after injection of an RGD-modified hydrogel containing autologous ovine MSC.

FIG. 12 illustrates in vivo micro-computed tomography images of pelvic portions of sheep 12 weeks after injection of an RGD-modified hydrogel containing autologous ovine MSC.

FIG. 13 illustrates 3-dimensional in vivo micro-computed tomography images of pelvic portions sheep 12 weeks after injection of an RGD-modified hydrogel containing autologous ovine MSC.

FIG. 14 illustrates graphs depicting the bone mineral density and bone volume fraction of sheep 12 weeks after injection of an RGD-modified hydrogel compared to an acellular gel.

### SUMMARY

The invention provides a hydrogel composition and methods of producing the hydrogel composition. The hydrogel composition comprises an alginate polymer and a hyaluronic acid polymer. A cell-instructive peptide is covalently linked to at least a portion of the alginate polymer and/or the hyaluronic acid polymer. In some embodiments, the composition comprises 0.5%-5% alginate polymer, for example 2% alginate polymer. In some embodiments, the composition comprises 0.25%-5% hyaluronic acid polymer, for example 0.5% or 1% hyaluronic acid polymer.

The cell-instructive peptide may be RGD (Arg-Gly-Asp), YIGSR (Tyr-Ile-Gly-Ser-Arg), IKVAV (Ile-Lys-Val-Ala-Val), GFOGER (GGYGGGPC(GPP)5GFOGER(GPP)5GPC), or GHK (Gly-His-Lys), for example. In an embodiment, the cell-instructive peptide is an adhesion peptide. In another embodiment, the hydrogel composition further comprises a mineralized polymeric microsphere. In further embodiments, the polymeric microsphere comprises polylactic acid (PLA), polyglycolide (PGA), poly(lactic-co-glycolic acid) (PLGA), or polycaprolactone (PCL). In a further embodiment, the polymeric microsphere is coated with a ceramic, apatite, or bone-like mineral. In another embodiment, the hydrogel composition further comprises a fibrin hydrogel and an apatite-coated mineralized polymeric microsphere.

In an embodiment, the hydrogel composition comprises an alginate polymer, the composition comprising 0.5%-5% alginate polymer; a hyaluronic acid polymer, the composition comprising 0.25%-5% hyaluronic acid polymer; and a mineralized polymeric microsphere, wherein a cell-instructive peptide is covalently linked to the alginate polymer and the hyaluronic acid polymer, and wherein the cell-instructive peptide is an adhesion peptide.

The hydrogel composition may be produced by covalently coupling the cell-instructive peptide with hyaluronic acid (HA) and alginate, mixing the HA-cell-instructive peptide and alginate-cell-instructive peptide components together to form a solution, adding a polymeric microsphere to form a second solution, and gelling the second solution to form the hydrogel composition.

The invention also provides a method of administering the hydrogel composition to a subject. In an embodiment, the method includes administering the hydrogel composition to the subject.

### DETAILED DESCRIPTION

The figures and the following description relate to various embodiments of the invention by way of illustration only. It should be noted that from the following discussion, alternative embodiments of the structures and methods disclosed herein will be readily recognized as viable alternatives that may be employed without departing from the principles of what is claimed.

It must be noted that, as used in the specification and the appended claims, the singular forms “a,” “an” and “the” include plural referents unless the context clearly dictates otherwise.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Compositions for a hydrogel composition composed of alginate, HA, and cell-instructive peptides, as well as methods for administering the hydrogel composition to a subject to repair tissue defects, are disclosed.

Alginate is an anionic polysaccharide, which is a linear copolymer. Alginate is highly cytocompatible and resorbs slowly, and functions well as an injectable material that gels in the presence of ionic moieties such as calcium and barium. A gel composed of alginate breaks down slowly in vivo, but gels in the presence of calcium. Thus, the viscosity of this gel can be controlled by the molecular weight of the polymer, the concentration of calcium in the gel, or the percentage of guluronic acid present in the alginate polymer. Hyaluronic acid (HA) is an anionic glycosaminoglycan, found in connective tissues and at healing sites. HA can be administered as an adjuvant to stimulate tissue repair. A gel composed of HA is relatively not viscous, and requires alternative chemistries to promote gelation. The combination of alginate with HA is useful for producing an injectable hydrogel system that enables a controllable gelation time and stimulates tissue repair while degrading into resorbable components. However, neither alginate nor HA enable cell adhesion or contain appropriate binding sites for proteins that enable cell adhesion.

The invention provides a hydrogel composition produced by chemically grafting cell-instructive peptides on the backbone of HA and/or alginate, and combining the HA-cell-instructive peptide and alginate-cell-instructive peptide components into a hydrogel system. A cell-instructive peptide is a sequence of amino acids that is responsible for some change in a cell. In an embodiment, the cell-instructive peptide promotes secretion of endogenous signals by cells engaging the peptide. In an embodiment, the cell-instructive peptide is GHK (Gly-His-Lys), which causes cells to secrete proteins that stimulate vascularization. In another embodiment, the cell-instructive peptide is an adhesion peptide. Adhesion peptides aid in the binding of cells to a surface, enabling cell adhesion and promoting cell survival. One example of an adhesion peptide is the RGD sequence (Arg-Gly-Asp), which is included in fibronectin. Other examples include YIGSR (Tyr-Ile-Gly-Ser-Arg), IKVAV (Ile-Lys-Val-Ala-Val), and GFOGER (GGYGGGPC(GPP)5GFOGER(GPP)5GPC). In an embodiment, the chemical grafting process of adhesion peptides to HA and/or alginate is performed using standard carbodiimide chemistry (Rowley et al., Biomaterials 1999).

These hydrogel compositions may be injected into a subject at a tissue defect site in order to stimulate tissue repair. Depending on the composition of the hydrogel, the speed of gelation and resorption time in situ can be controlled. In one embodiment, the HA-cell-instructive peptide and/or alginate-cell-instructive peptide components are purified to eliminate ungrafted peptide and lyophilization, and all materials are resuspended in basal cell culture medium and mixed at varying mass ratios to control gelation time and in situ resorption. In one embodiment, gels with 2% alginate and 0.5% HA degrade more slowly than gels with 2% alginate and 1% HA. Gels with a higher concentration of alginate are relatively stiff, but also relatively more viscous and difficult to inject. In one embodiment, a gel can include 0.25%-5% hyaluronic acid polymer. In further embodiments, the gel can include 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, or 5% hyaluronic acid. In another embodiment, the gel can include 0.5%-5% alginate polymer. In further embodiments, the gel can include 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, or 5% alginate polymer. In another embodiment, the gel can include 1%-4% alginate polymer.

In an embodiment, cell suspensions such as mesenchymal stem cells in basal cell culture medium may be mixed with alginate, HA, and gelation solution (such as CaSO₄, CaCl₂, or other calcium sources) to produce a hydrogel, to a concentration of 1-100 million cells/mL. In a further embodiment, 0.5-1.5 mL of the mixture is injected into a subject. In further embodiments, the subject is a horse, a dog, or a cat. In some embodiments, the hydrogel is not injected into the subject, but instead is administered to a subject as an implantable disc or plug.

In one embodiment, the hydrogel compositions may also include nonmineralized and/or mineralized biodegradable polymer microspheres, which allow for improved distribution throughout the hydrogel and improved spatial interaction with cells, and enhance construct osteoconductivity and osseointegration within a bone tissue defect site. Polymeric microspheres are fabricated from synthetic polymers such as poly(lactide), poly(glycolide), and co-polymers thereof, as well as other polymeric materials using a double emulsion or extrusion process. Microspheres can be prepared using various methods (see Example 1). In some embodiments, microspheres are hydrolyzed to functionalize the polymer surface, placed in a modified simulated body fluid (mSBF) and incubated at a temperature between 20° C.-40° C. for 0.5-10 days, making sure to exchange the solution periodically to maintain appropriate ion concentrations. This process is called “biomineralization.” Other available methods of biomineralization rapidly deposit carbonated apatite on the polymer surface. Furthermore, polymers may include composites containing calcium phosphate-based ceramics (hydroxyapatite, beta-tri-calcium phosphate, orthophosphate, brushite, etc.).

After the microspheres are prepared, they can be suspended in hydrogels by adding 1-35 mg/mL microspheres in a hydrogel.

Unlike other approaches which require the addition of large masses of bioceramics or the prolonged incubation in simulated body fluid (thus eliminating the injectable aspect of delivery), in some embodiments prefabricated polymer microspheres are used that remain suspended in the gel, can pass through a range of needle sizes, and nucleate calcium within the implant.

The methods described herein are useful, e.g, for manufacture of implantable materials to guide tissue formation or produce 3D substrates for the study of cellular behavior. These methods have the ability to accelerate bone formation, enhance biomaterial integration once implanted, and provide a platform to present proteins in a more controlled and localized manner in vivo.

Benefits include less construct degradation, more flexibility, retention of injectability, and uniform distribution in the gel. This would also allow for controlled drug presentation from the microspheres that could be generated from many synthetic polymers that can nucleate mineral, including polylactic acid (PLA), polyglycolide (PGA), poly(lactic-co-glycolic acid) (PLGA), and polycaprolactone (PCL).

### EXAMPLES

The following Examples illustrate methods of producing hydrogel compositions composed of alginate, HA, and cell-instructive peptides, as well as methods of administering the hydrogel compositions at sites in need of tissue repair.

### Example 1

### Preparation of Polymeric Microspheres

Polymeric microspheres were formed using a standard double emulsion technique (Cohen, S., Yoshioka, T., Lucarelli, M., Hwang, L. H., and Langer, R. Controlled delivery systems for proteins based on poly(lactic glycolic acid) microspheres. PharmRes 8, 713, 1991). To prepare mineralized polymeric substrates, microspheres were hydrolyzed for 10 min in 0.5M NaOH to functionalize the polymer surface, and rinsed in distilled H₂O. Microspheres were immediately placed in modified simulated body fluid (mSBF), incubated at 37° C. for 7 days (making sure to exchange the solution daily to maintain appropriate ion), frozen overnight at −80° C., and lyophilized for three days. The mSBF consisted of the following reagents dissolved in DI H2O: 141 mM NaCl, 5.0 mM CaCl₂, 4.2 mM NaHCO₃, 4.0 mM KCl, 2.0 mM KH₂PO₄, 1.0 mM MgCl₂, and 0.5 mM MgSO₄. The solution was held at pH 6.8 to avoid homogeneous precipitation of CaP phases. Both nonmineralized and mineralized microspheres were strained through a testing sieve to collect particles with diameters less than 250 μm to avoid microsphere clumping. Microspheres were sterilized under ultraviolet light for 15 minutes prior to use.

Microspheres were then sterilized in ethanol and lyophilized until dry, keeping them sterile and ready for combination with any injectable system.

### Example 2

### An Injectable RGD-Coupled Composite Alginate Hydrogel for Large Bone Defect Repair

### Materials

Ultra pure PRONOVA UP MVG sodium alginate with an average molecular weight (MWs)>200 kDa was from FMC Biopolymer (Princeton, N.J.). PLG (poly(D,L-lactide-co-glycolide)) pellets with a copolymer ratio of 85:15 [lactic:glycolic DL (dextrorotary and levorotary isomers) (%)] and MWs were from Lakeshore Biomaterials (Birmingham, Ala., USA). Polyvinylalcohol (PVA) of average MWs 9000-10000, 88% hydrolyzed was from Aldrich chemical Co. Modified simulated body fluid (mSBF) was prepared as previously described (Murphy W L, et al. J Biomed Mater Res. 2000; Davis H E et al., Biotechnol Bioeng. 2011; Davis H E et al. J Biomed Mater Res A. 2009) and consisted of the following reagents dissolved in distilled H₂O: 141 mM NaCl, 5.0 mM CaCl₂, 4.2 mM NaHCO₃, 4.0 mM KCl, 2.0 mM KH₂PO₄, 1.0 mM MgCl₂, and 0.5 mM MgSO₄. The solution was held at pH=6.8 to avoid homogeneous precipitation of CaP phases.

### Irradiation and RGD Coupling

Sodium alginate (FMC Biopolymer, Princeton, N.J.) was subjected to a 5 Mrad dose of gamma irradiation for faster degradation. The irradiated alginates were then covalently coupled with G₄RGDSP peptide sequences using standard carbodiimide chemistry. Irradiation shortens the polymer chain lengths and increases the speed of degradation. In some embodiments, oxidation through incubation in sodium periodate (Kong et al., Biomacromolecules (2004)) is performed instead of irradiation. In other embodiments, oxidation and irradiation methods are combined to further accelerate degradation. The resulting RGD-alginates were sterile filtered, lyophilized and stored at −20° C. Similarly, sodium hyaluron (HA) was treated identically to couple with RGD peptide sequence by carbodiimide chemistry.

### Preparation of Cellular Constructs

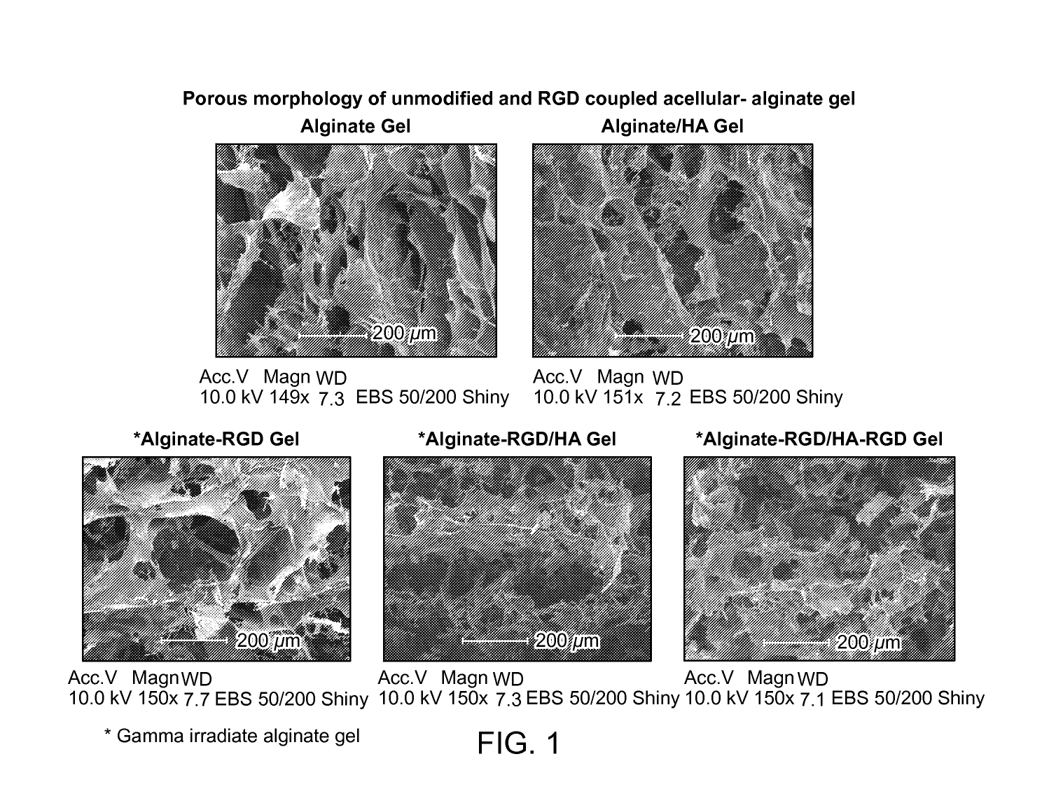

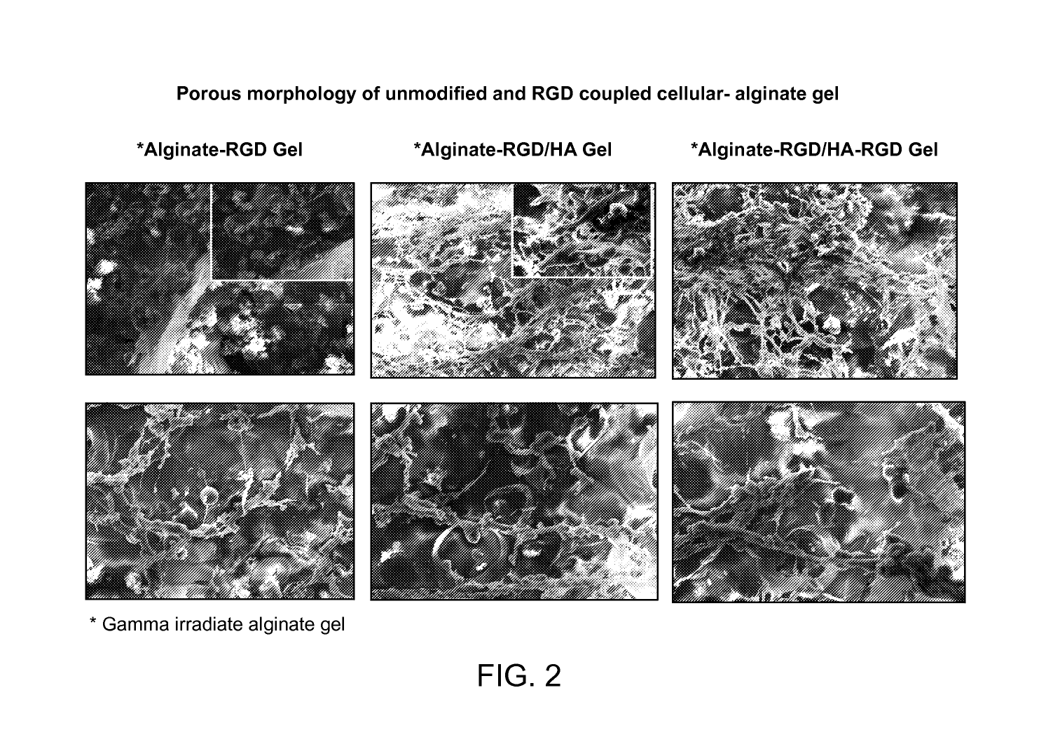

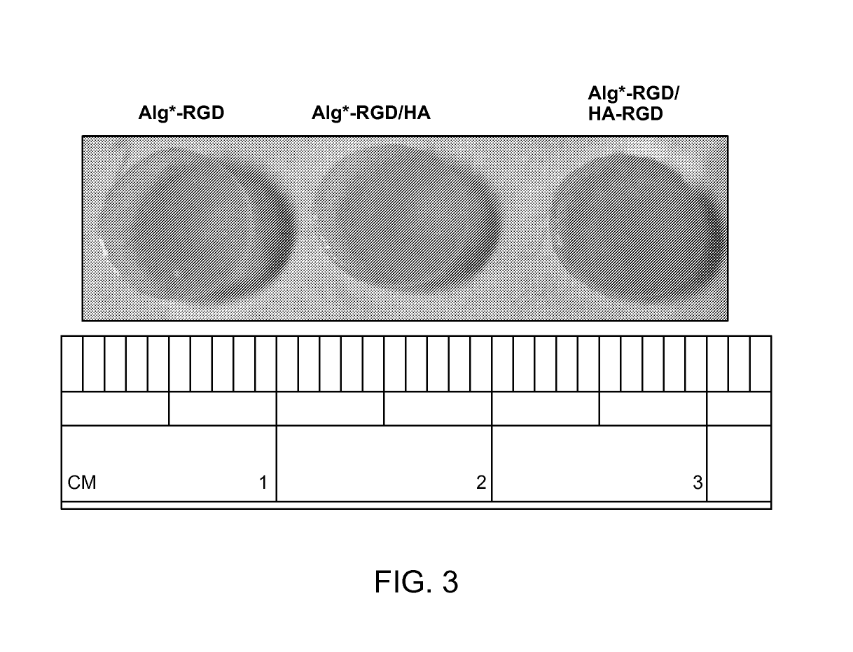

To prepare hydrogels, the alginate-RGD and HA-RGD were reconstituted separately in α-MEM to obtain a 2% (w/v) of alginate and 1% (w/v) of HA-RGD solutions (FIG. 1 and FIG. 2). Sterile 0.22 μm filtered RGD-coupled alginate solution was mixed with sterile filtered RGD-coupled HA solution at 9:1 (v/v) ratio with UV sterilized mineralized microspheres (MM) at a concentration of 3 mg/mL in micro-centrifuge tube. This solution was then mixed with 100 μL cell suspension in α-MEM with a seeding density of 10 million cells/mL. Alginate-RGD/HA-RGD solution containing cells and mineralized microspheres was cross-linked with calcium sulfate slurry (0.21 g CaSO₄ per 1 mL deionized water) at a ratio of 17:1 (50 μL of CaSO₄ with 850 μL of alginate-RGD/HA-RGD/MM solution). The mixing was performed in two 1-mL syringes (Becton-Dickinson, Franklin Lakes, N.J.) coupled with a 3-way stopcock syringe connector (Smith Medical ASD Inc., Dublin, Ohio) with Luer-Lok fittings to minimize air bubbles, with the resulting gels as shown (FIG. 1, FIG. 2, and FIG. 3).

The pre-gelled hydrogel solution mixing was performed aseptically in two 1-mL syringes coupled with a 3-way stopcock syringe connector and approximately 1000-1500 μL pre-gelled hydrogel samples injected in the defect core using a 21 g needle (Becton-Dickinson, Franklin Lakes, N.J.) attached to one of the syringes used for gel mixing.

### Example 3

### Repairing Iliac Wing Defects in Sheep Using RGD-Coupled Composite Alginate Hydrogel

### Materials

Ultra pure PRONOVA UP MVG sodium alginate with an average MWs>200 kDa was from FMC Biopolymer (Princeton, N.J.). PLG pellets with a copolymer ratio of 85:15 [lactic:glycolic DL (%)] and MWs were from Lakeshore Biomaterials (Birmingham, Ala., USA). Polyvinylalcohol (PVA) of average MWs 9000-10000, 88% hydrolyzed was from Sigma Aldrich. Modified simulated body fluid (mSBF) was prepared as previously described (Murphy W L, et al. J Biomed Mater Res. 2000; Davis H E et al., Biotechnol Bioeng. 2011; Davis H E et al., J Biomed Mater Res A. 2009) and consisted of the following reagents dissolved in distilled H₂O: 141 mM NaCl, 5.0 mM CaCl₂, 4.2 mM NaHCO₃, 4.0 mM KCl, 2.0 mM KH₂PO₄, 1.0 mM MgCl₂, and 0.5 mM MgSO₄. The solution was held at pH=6.8 to avoid homogeneous precipitation of CaP phases. Human MSCs were purchased from Lonza (Walkersville, Md., USA) at passage 2 and expanded in αMEM (Invitrogen, Carlsbad, Calif., USA) supplemented with 10% fetal bovine serum (JR Scientific, Woodland, Calif., USA) and 1% penicillin/streptomycin (Mediatech) until use at passage 4-6.

### Irradiation and RGD Coupling

Mineralized microspheres were fabricated as described above.

Sodium alginate (FMC Biopolymer, Princeton, N.J.) was subjected to a 5 Mrad dose of gamma irradiation for faster degradation. The irradiated alginates were then covalently coupled with G₄RGDSP peptide sequences (Celtek Petides, Nashville, Tenn.) using standard carbodiimide chemistry. The resulting RGD-alginates were sterile filtered, lyophilized and stored at −20° C. Similarly, sodium hyaluronan (HA) was treated identically to couple with RGD peptide sequence by carbodiimide chemistry.

### Preparation of Cellular and Acellular Gel Constructs

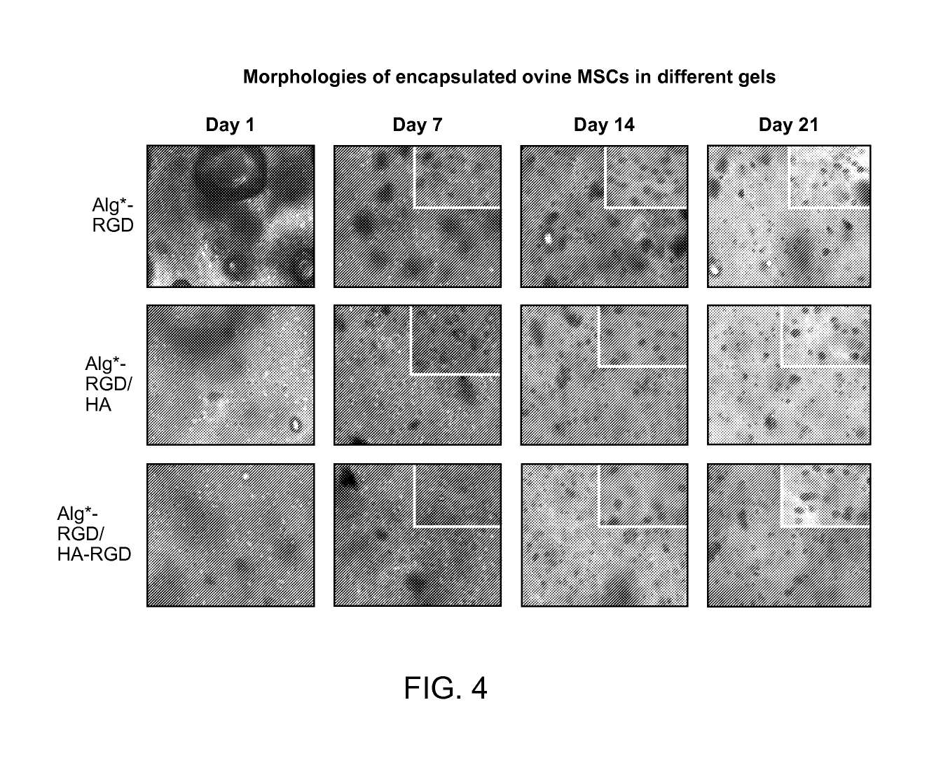

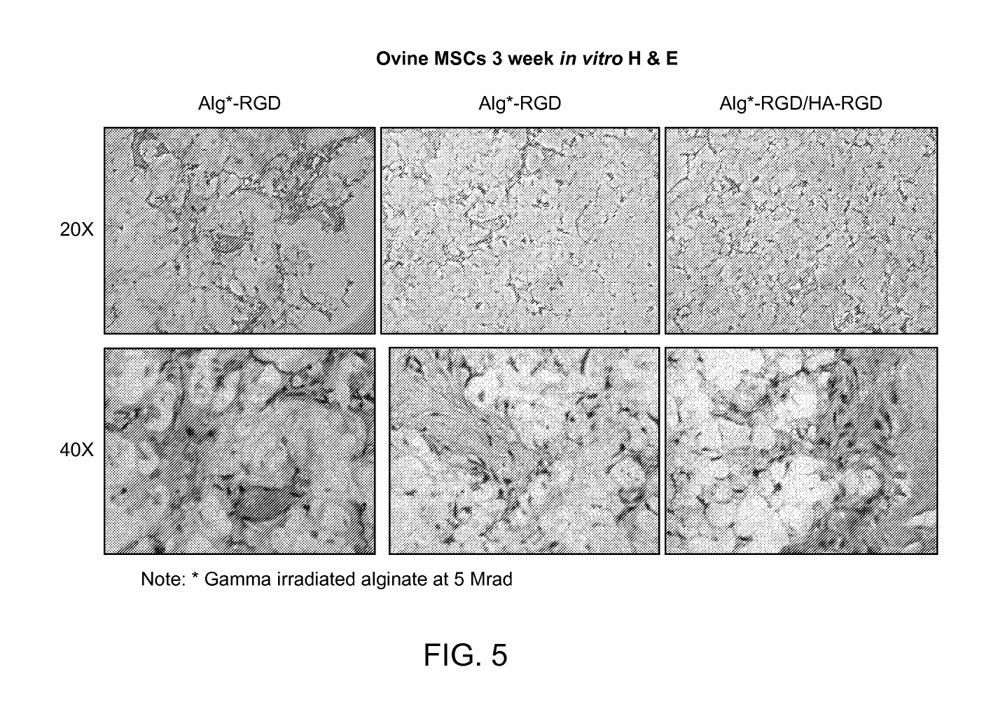

To prepare hydrogels, the alginate-RGD and HA-RGD were reconstituted separately in α-MEM to obtain a 2% (w/v) of alginate and 1% (w/v) of HA-RGD solutions. Sterile 0.22 μm filtered RGD-coupled alginate solution was mixed with sterile filtered RGD-coupled HA solution at 9:1 (v/v) ratio with UV sterilized mineralized microspheres (MM) at a concentration of 3 mg/mL in micro-centrifuge tube. This solution was then mixed with 100 μL oMSCs suspension in α-MEM with a seeding density 10 million cells/mL (FIG. 4 and FIG. 5). Alginate-RGD/HA-RGD solution containing cells and mineralized microspheres was cross-linked with calcium sulfate slurry (0.21 g CaSO₄ per 1 mL deionized water) at a ratio of 17:1 (50 μL of CaSO₄ with 850 μL of alginate-RGD/HA-RGD/MM solution). The mixing was performed in two 1-mL syringes (Becton-Dickinson, Franklin Lakes, N.J.) coupled with a 3-way stopcock syringe connector (Smith Medical ASD Inc., Dublin, Ohio) with Luer-Lok fittings to minimize air bubbles. The gelling alginate-RGD/HA-RGD solution containing cells and MM was dispensed between sterile parallel glass plates with 1 mm spacers. The alginate-RGD/HA-RGD was allowed to gel for 45-60 min in incubator, and circular gel disks were cut out with 8 mm biopsy punches. The mineralized microspheres loaded cellular alginate-RGD/HA-RGD disks/constructs were then transferred in each 24-well plates and cultured for 3 weeks at 37° C. with 5% CO₂ in 0.5 mL osteogenic α-MEM medium supplemented with 10% FBS, 1% P/S, 10 mM β-glycerophosphate, 50 μg/mL ascorbate-2-phosphate and 10 nM dexamethasone, which was replaced every 2-3 days. The second set of cellular MM loaded alginate-RGD hydrogel was prepared in combination with unmodified HA (Alg-RGD/HA), while the third set of cellular MM loaded Alginate-RGD (Alg-RGD) was prepared alone by the same procedure as described above. Aseptic conditions were maintained in all the above steps, including handling of the exterior of the syringe.

Similarly, acellular Alginate-RGD/HA-RGD, Alginate-RGD/HA, and Alginate-RGD containing MM (mineralized microspheres) were synthesized non-aseptically using the above procedure, which was done for the purpose of characterizing mechanical and swelling performances of these gel constructs. Using a 5 mm biopsy punch, gels (5 mm diameter and 2 mm height thickness) were then cut from hydrogel sheet and allowed to equilibrate in deionized (DI) water for at least 24 h before use.

### Swelling Degree Measurement

Gels were placed in excess DI water for at least 24 h to remove extractable materials from the polymer networks. Equilibrated cell-free gel samples were weighed and dried in lyophilizer. After at least 48 h, the dried gel samples were removed and weighed again. The swelling degree, Q, was calculated as the ratio of the weight of the equilibrated hydrogel samples to its dry weight.

### Mechanical Testing

The compressive modulus of the RGD-coupled alginate hydrogels was determined at room temperature on Instron-3345 compressing testing system (Norwood, Mass., USA). Gels were equilibrated in PBS for 24 hours at room temperature prior to measurement. Any excess fluid was blotted off from the gel surface before analysis and then loaded between two flat platens. Hydrogel disks (5 mm dia & 2 mm thickness, n=5 per group) were then compressed with a 10N loading cell between two flat platens in the direction normal to the circular face of the gel at a rate of 1 mm/min. The compressive elastic modulus, defined as the slope of the linear region of the stress-strain curve of a material under compression, was calculated from the initial linear portion of the curve (0-5% strain).

### Morphological Characterization of Composite Hydrogel

The morphological characteristics of composite MM (mineralized microsphere) loaded alginate gels were observed by scanning electron microscopy. To examine the surface morphology and internal structure of alginate hydrogel, samples were cut with a scalpel blade and were sputter-coated using a Pelco Auto Sputter Coater SC-7 (Ted Pella Inc., Redding, Calif.) at 20 mA with gold for 30 seconds prior to imaging. The morphology of the hydrogels was evaluated by Philips XL30 TMP field emission Scanning Electron Microscope (FEI Company, Eindhoven, Netherlands). The pore diameter of the fibers was quantified by analyzing the SEM images at 150× and 300× magnification.

### In Vitro Osteogenic Response

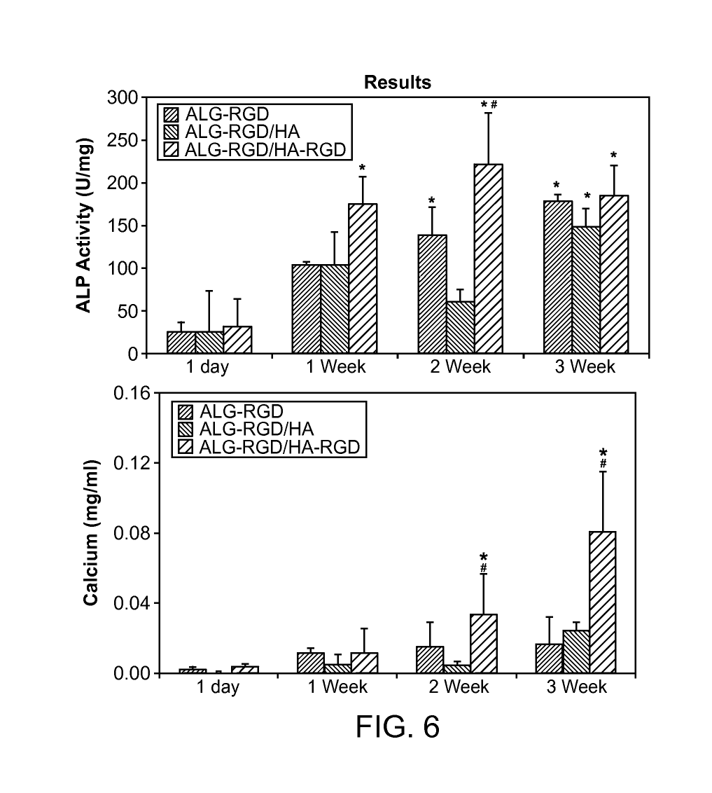

The Alg-RGD, Alg-RGD/HA and Alg-RGD/HA-RGD gels samples (n=4 per group) were subsequently rinsed with PBS (Sigma) and collected in 400 μL of passive lysis buffer (Promega, Madison, Wis.). Immediately following one freeze-thaw cycle, lysates were sonicated briefly, centrifuged for 5 minutes at 10,000 rpm, and the supernatant was used to determine DNA content, calcium content, and intracellular alkaline phosphatase activity (ALP). Total DNA present in each hydrogel construct was quantified using the Quant-iT PicoGreen dsDNA kit (Invitrogen) in comparison to a known standard curve. Intracellular alkaline phosphatase (ALP) from oMSCs seeded alginate scaffolds was quantified using a PNPP colorimetric assay at 405 nm as described (He, J., Genetos, D. C., Yellowley, C. E., and Leach, J. K. J Cell Biochem 110, 87, 2010). ALP activity was normalized to DNA content determined as described above (FIG. 6). Total calcium present on oMSC-seeded hydrogel scaffolds was measured using an OCOP colorimetric assay similar to that previously described (He, J., Genetos, D. C., Yellowley, C. E., and Leach, J. K. J Cell Biochem 110, 87, 2010). Briefly, minced hydrogel disks were incubated in 0.9N H₂SO₄ overnight to solubilize surface calcium deposits. Calcium concentration in solution was then quantified and compared to a known standard curve. To account for calcium present in the MM loaded gel samples, calcium in acellular gels was quantified at each time point and subtracted from calcium values obtained from gels containing cells at each time point (FIG. 6).

### In Vivo Surgical Procedure and Analysis

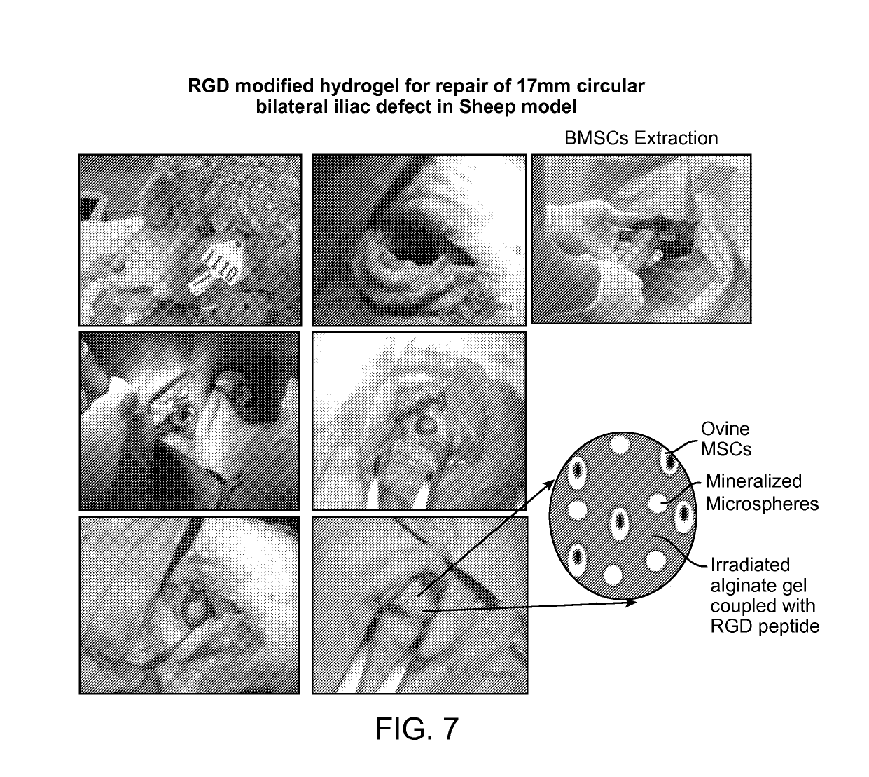

A critical-sized bilateral 17 mm circular and 5 mm long defect was created in the iliac wing of 4-6 year old sheep (n=6) according to a protocol approved by the Institutional Animal Care and Use Committee (IACUC) at the University of California, Davis (FIG. 7). 12 male sheep of age 4-6 years were used for the study. Defects were created with a 17 mm drill bit utilizing a custom made jig fixed in position using the locating screws placed during surgery to ensure the correct location. The pre-gelled hydrogel solution mixing was performed outside aseptically in two 1-mL syringes coupled with a 3-way stopcock syringe connector and approximately 1000-1500 μL pre-gelled hydrogel samples injected in the defect core using a 21 g needle (Becton-Dickinson, Franklin Lakes, N.J.) attached to one of the syringes used for gel mixing. In control group (n=6) one side of defects were immediately filled with MM (mineralized microsphere) loaded Alg-RGD/HA-RGD hydrogel alone, while contra lateral wing were left as unfilled (Sham/empty). In experimental group (n=6) defects were filled with MM (mineralized microsphere) loaded Alg-RGD/HA-RGD hydrogel with autologous MSCs (10 millions cells/mL), while contra lateral wing were implanted with MM loaded Alg-RGD/HA-RGD hydrogel which served as the negative control.

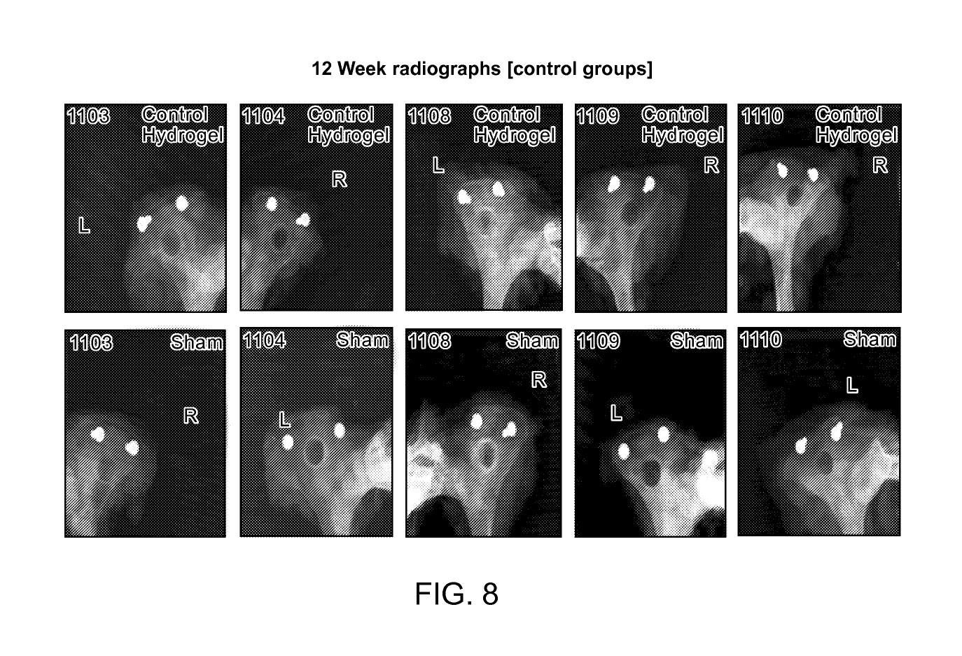

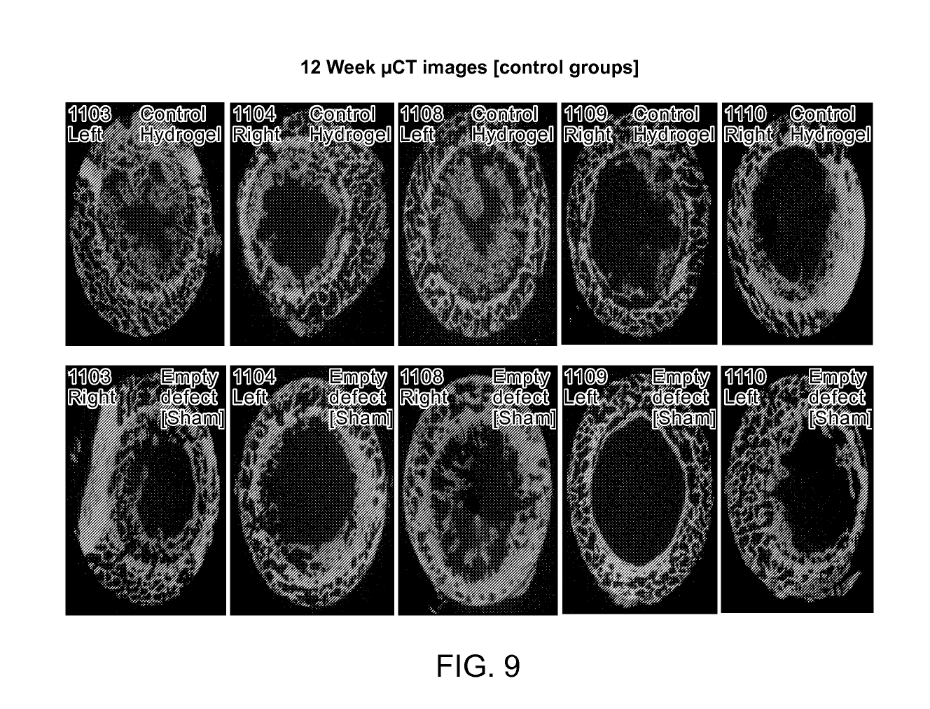

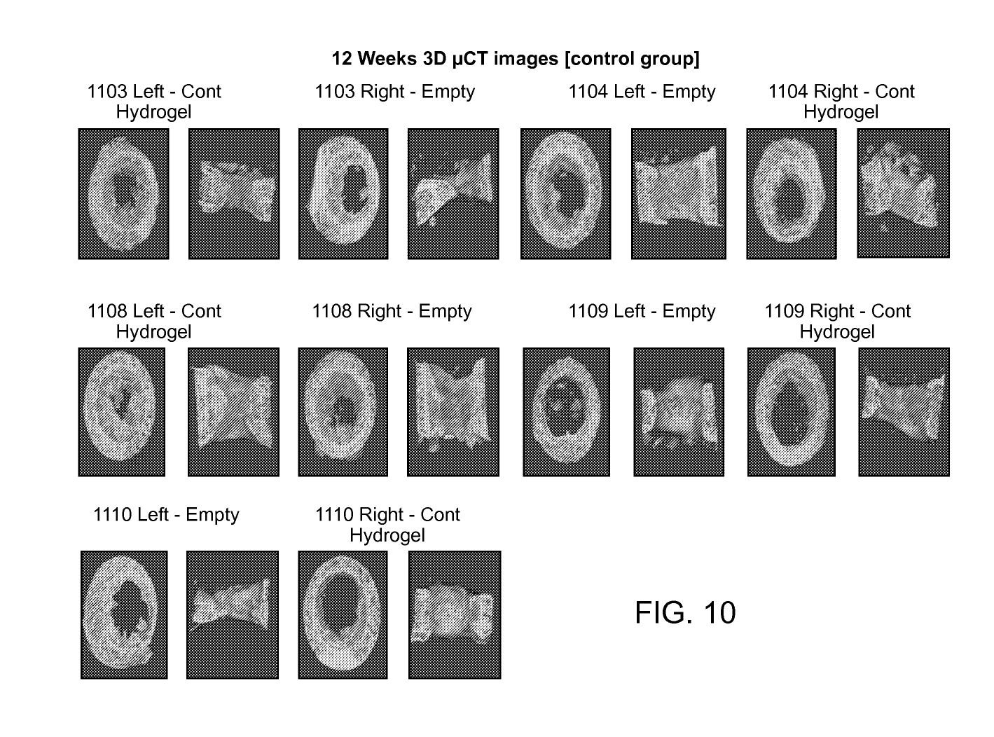

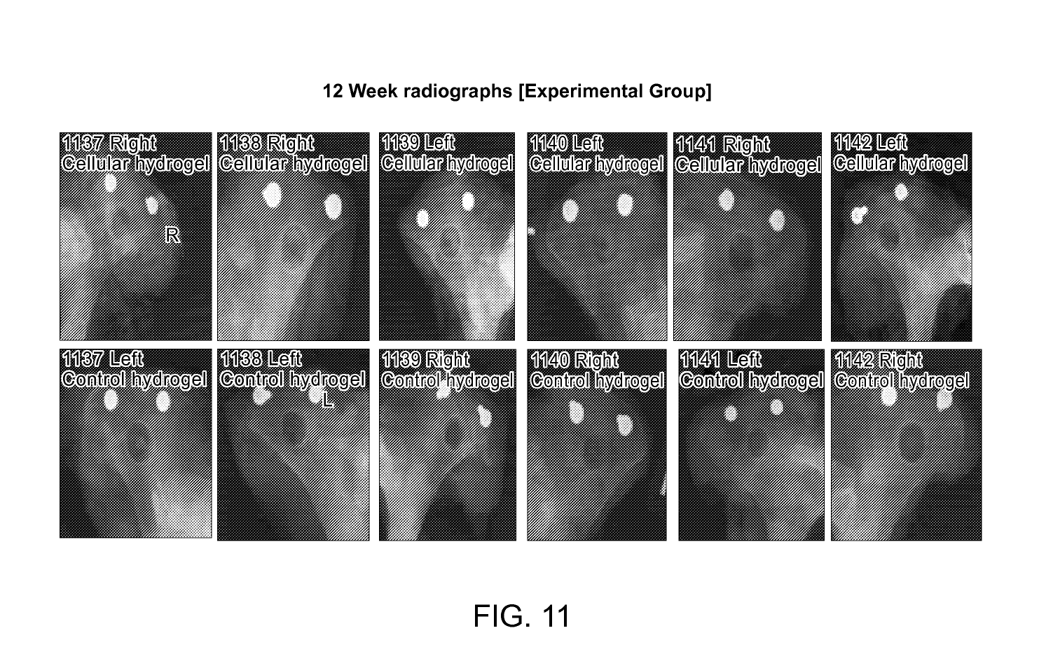

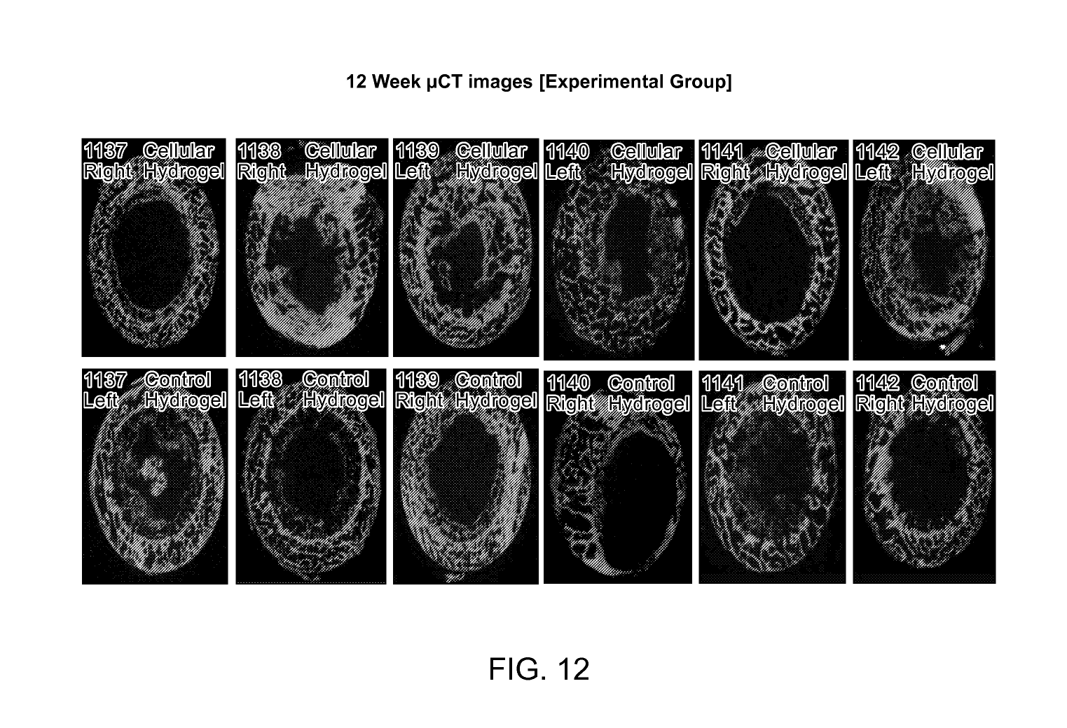

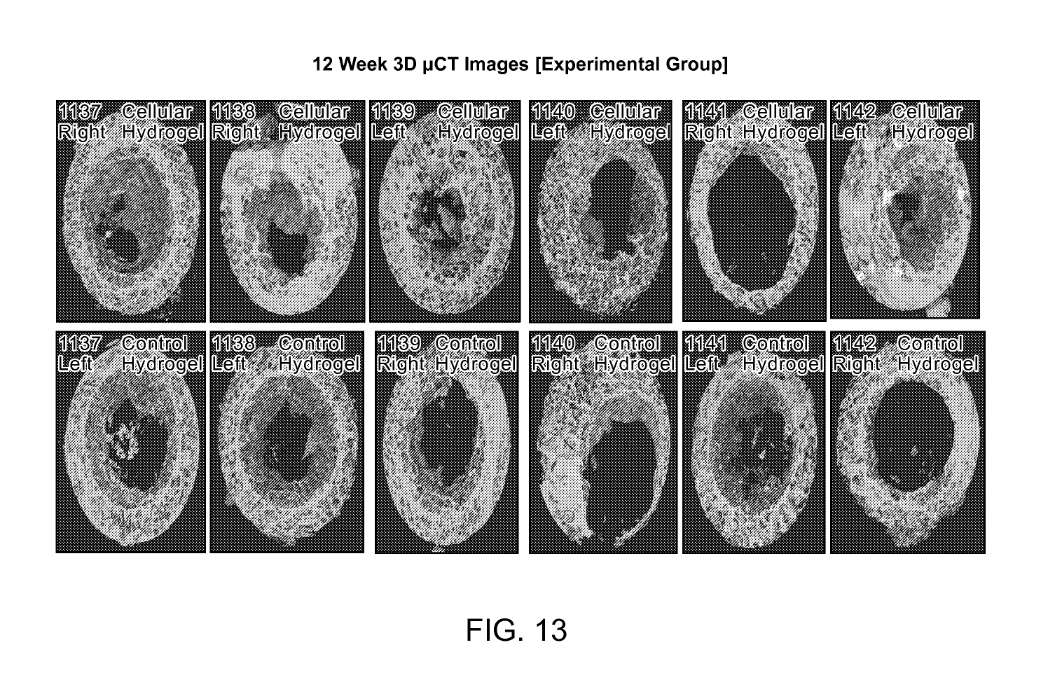

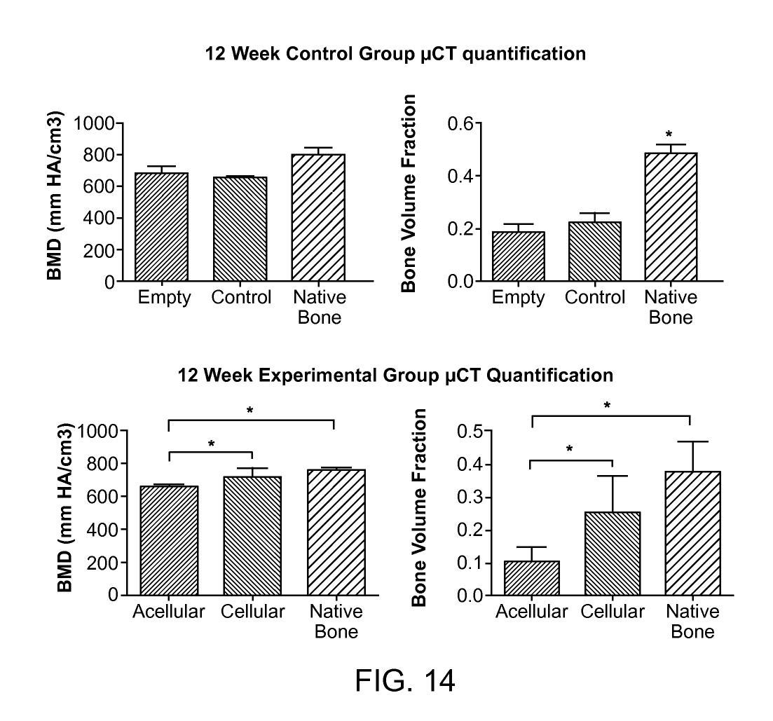

The groups were assigned to the right and left iliac wings to evenly distribute pairs of groups and obtain a balanced experimental design. The periosteum, muscle, and skin were sutured over the iliac crest in layers immediately after the defect filling. After surgery, the sheep were allowed to recover and move freely. Radiographs (2, 10 and 12 weeks after surgery) and in vivo microcomputed tomography (XT) images (12 weeks after surgery) were obtained to evaluate bone healing (FIGS. 8-13). Sheep were humanely euthanized by a high dose of intravenous barbiturate at 12 weeks after surgery. Immediately after euthanasia, the pelvis were extracted for radiographs and the healed defects were cored out with a 25 mm drill bit utilizing a custom made jig fixed in position using the locating screws placed during surgery. Samples were fixed in 10% neutral-buffered formalin for 48 hours and preserved in 70% ethanol for μCT and histological analysis. Significantly more bone was formed in iliac wing defects treated with cellularized hydrogels compared to acellular gels (FIG. 14). The treated defects had significantly more bone ingrowth from the periphery that penetrated toward the center.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

All references, issued patents and patent applications cited within the body of the instant specification are hereby incorporated by reference in their entirety, for all purposes.

### REFERENCES

1. Victor W. M H, Annemie C, Pieter K. Role of fibrin matrix in angiogenesis Ann N Y Acad Sci. 936:426-37. 2001.
2. Janmey P A, Winer J P, Weisel J W. Fibrin gels and their clinical and bioengineering applications. J R Soc Interface. 6:1-10. 2009.
3. Breen A, O'Brien T, Pandit A. Fibrin as a delivery system for therapeutic drugs and biomolecules. Tissue Eng Part B-Rev. 15:201-14. 2009.
4. Ahmed T A E, Dare E V, Hincke M. Fibrin: A versatile scaffold for tissue engineering applications. Tissue Eng Part B-Rev. 14:199-215. 2008.
5. Zhao H G, Ma L, Zhou J, Mao Z W, Gao C Y, Shen J C. Fabrication and physical and biological properties of fibrin gel derived from human plasma. Biomed Mater. 3:9. 2008.
6. Yamada Y, Seong Boo J, Ozawa R, Nagasaka T, Okazaki Y, Hata K-i, et al. Bone regeneration following injection of mesenchymal stem cells and fibrin glue with a biodegradable scaffold. J Craniomaxillofac Surg. 31:27-33. 2003.
7. Dickneite G, Metzner H, Pfeifer T, Kroez M, Witzke G. A comparison of fibrin sealants in relation to their in vitro and in vivo properties. Thromb Res. 112:73-82. 2003.
8. Ho W, Tawil B, Dunn J C Y, Wu B M. The behavior of human mesenchymal stem cells in 3D fibrin clots: dependence on fibrinogen concentration and clot structure. Tissue Eng. 12:1587-95. 2006.
9. Duong H, Wu B, Tawil B. Modulation of 3D fibrin matrix stiffness by intrinsic fibrinogen and thrombin compositions and by extrinsic cellular activity. Tissue Eng Part A. 15:1865-76. 2009.
10. Catelas I, Sese N, Wu B M, Dunn J C Y, Helgerson S, Tawil B. Human mesenchymal stem cell proliferation and osteogenic differentiation in fibrin gels in vitro. Tissue Eng. 12:2385-96. 2006.
11. Davis H E, Miller S L, Case E M, Leach J K. Supplementation of fibrin gels with sodium chloride enhances physical properties and ensuing osteogenic response. Acta Biomater. 7:691-9. 2011.
12. Davis H, Leach J. Designing bioactive delivery systems for tissue regeneration Ann Biomed Eng. 39:1-13. 2011.
13. Lee K, Silva E A, Mooney D J. Growth factor delivery-based tissue engineering: general approaches and a review of recent developments. J R Soc Interface. 8:153-70. 2011.
14. Malda J, Frondoza C G. Microcarriers in the engineering of cartilage and bone. Trends Biotechnol. 24:299-304. 2006.
15. Jongpaiboonkit L, Franklin-Ford T, Murphy W L. Growth of hydroxyapatite coatings on biodegradable polymer microspheres. ACS Appl Mater Interfaces. 1:1504-11. 2009.
16. Jongpaiboonkit L, Franklin-Ford T, Murphy W L. Mineral-coated polymer microspheres for controlled protein binding and release. Adv Mater. 21:1960-3. 2009.
17. Davis H E, Rao R R, He J, Leach J K. Biomimetic scaffolds fabricated from apatite-coated polymer microspheres. J Biomed Mater Res A. 90:1021-31. 2009.
18. Kim S S, Gwak S J, Kim B S. Orthotopic bone formation by implantation of apatite-coated poly(lactide-co-glycolide)/hydroxyapatite composite particulates and bone morphogenetic protein-2. J Biomed Mater Res A. 87:245-53. 2008.
19. Haiguang Z, Lie M, Changyou G, Jinfu W, Jiacong S. Fabrication and properties of injectable beta-tricalcium phosphate particles/fibrin gel composite scaffolds for bone tissue engineering. Mater Sci Eng C Mater Biol Appl. 29:836-42. 2009.
20. Le Nihouannen D, Le Guehennec L, Rouillon T, Pilet P, Bilban M, Layrolle P, et al. Micro-architecture of calcium phosphate granules and fibrin glue composites for bone tissue engineering. Biomaterials. 27:2716-22. 2006.
21. Osathanon T, Linnes M L, Rajachar R M, Ratner B D, Somerman M J, Giachelli C M. Microporous nanofibrous fibrin-based scaffolds for bone tissue engineering. Biomaterials. 29:4091-9. 2008.
22. Murphy W L, Kohn D H, Mooney D J. Growth of continuous bonelike mineral within porous poly(lactide-co-glycolide) scaffolds in vitro. J Biomed Mater Res. 50:50-8. 2000.
23. Davis H E, Case E M, Miller S L, Genetos D C, Leach J K. Osteogenic response to BMP-2 of hMSCs grown on apatite-coated scaffolds. Biotechnol Bioeng. 108:2727-35. 2011.
24. Cohen S, Yoshioka T, Lucarelli M, Hwang L H, Langer R. Controlled delivery systems for proteins based on poly(lactic glycolic acid) microspheres. Pharm Res. 8:713-20. 1991.
25. Even-Ram S. Fibrin gel model for assessment of cellular contractility. In: Even-Ram S, Artym V, eds. Extracellular Matrix Protocols: Humana Press; 2009. pp. 1-9.
26. Ghajar C M, Kachgal S, Kniazeva E, Mori H, Costes S V, George S C, et al. Mesenchymal cells stimulate capillary morphogenesis via distinct proteolytic mechanisms. Exp Cell Res. 316:813-25. 2010.
27. Leu A, Stieger S M, Dayton P, Ferrara K W, Leach J K. Angiogenic response to bioactive glass promotes bone healing in an irradiated calvarial defect. Tissue Eng Part A. 15:877-85. 2009.
28. He J, Decaris M L, Leach J K. Bioceramic-mediated trophic factor secretion by mesenchymal stem cells enhances in vitro endothelial cell persistence and in vivo angiogenesis. Tissue Eng Part A. 18:1520-8.2012.
29. He J, Genetos D C, Leach J K. Osteogenesis and trophic factor secretion are influenced by the composition of hydroxyapatite/poly(lactide-co-glycolide) composite scaffolds. Tissue Eng Part A. 16:127-37. 2010.
30. Cheung W K, Working D M, Galuppo L D, Leach J K. Osteogenic comparison of expanded and uncultured adipose stromal cells. Cytotherapy. 12:554-62. 2010.
31. Zhao H, Ma L, Gao C, Shen J. A composite scaffold of PLGA microspheres/fibrin gel for cartilage tissue engineering: Fabrication, physical properties, and cell responsiveness. J Biomed Mater Res B Appl Biomater. 88B:240-9. 2009.
32. Engler A J, Sweeney H L, Discher D E, Schwarzbauer J E. Extracellular matrix elasticity directs stem cell differentiation. J Musculoskelet Neuronal Interact. 7:335. 2007.
33. Engler A J, Sen S, Sweeney H L, Discher D E. Matrix elasticity directs stem cell lineage specification. Cell. 126:677-89. 2006.
34. Ratnoff O D, Potts A M. The accelerating effect of calcium and other cations on the conversion of fibrinogen to fibrin. J Clin Invest. 33:206-10. 1954.
35. Okada M, Blombäck B. Factors Influencing fibrin gel structure studied by flow measurement. Ann N Y Acad Sci. 408:233-53. 1983.
36. Okada M, Blomback B. Calcium and fibrin gel structure. Thromb Res. 29:269-80. 1983.
37. Murphy W L, Hsiong S, Richardson T P, Simmons C A, Mooney D J. Effects of a bone-like mineral film on phenotype of adult human mesenchymal stem cells in vitro. Biomaterials. 26:303-10. 2005.
38. Celil A B, Hollinger J O, Campbell P G. Osx transcriptional regulation is mediated by additional pathways to BMP2/Smad signaling. J Cell Biochem. 95:518-28. 2005.
39. Ling L, Nurcombe V, Cool S M. Wnt signaling controls the fate of mesenchymal stem cells. Gene. 433:1-7. 2009.
40. Wang C, Gong Y, Zhong Y, Yao Y, Su K, Wang D A. The control of anchorage-dependent cell behavior within a hydrogel/microcarrier system in an osteogenic model. Biomaterials. 30:2259-69. 2009.
41. Caplan A I, Dennis J E. Mesenchymal stem cells as trophic mediators. J Cell Biochem. 98:1076-84. 2006.
42. Hoch A I, Binder B Y, Genetos D C, Leach J K. Differentiation-dependent secretion of proangiogenic factors by mesenchymal stem cells. PLoS One. 7:e35579. 2012.
43. Lee J S, Suarez-Gonzalez D, Murphy W L. Mineral coatings for temporally controlled delivery of multiple proteins. Adv Mater. 23:4279-84. 2011.
44. Davis H E, Miller S L, Case E M, Leach J K. Supplementation of fibrin gels with sodium chloride enhances physical properties and ensuing osteogenic response. Acta Biomaterialia 2011; 7:691-699.
45. Davis H E, Rao R R, He, J, Leach J K. Biomimetic scaffolds fabricated from apatite-coated microspheres. Journal of Biomedical Materials Research Part A 2009; 90(4):1021-1031.
46. He, J., Genetos, D. C., Yellowley, C. E., and Leach, J. K. Oxygen tension differentially influences osteogenic differentiation of human adipose stem cells in 2D and 3D cultures. J Cell Biochem 110, 87, 2010.

1. A hydrogel composition comprising an alginate polymer and a hyaluronic acid polymer, wherein a cell-instructive peptide is covalently linked to at least a portion of the alginate polymer or the hyaluronic acid polymer.

2. A hydrogel composition, comprising:
an alginate polymer, the composition comprising 0.5%-5% alginate polymer;
a hyaluronic acid polymer, the composition comprising 0.25%-5% hyaluronic acid polymer; and
a mineralized polymeric microsphere,
wherein a cell-instructive peptide is covalently linked to the alginate polymer and the hyaluronic acid polymer, and wherein the cell-instructive peptide is an adhesion peptide.

3. The hydrogel composition of claim 1, wherein the cell-instructive peptide is covalently linked to the alginate polymer and the hyaluronic acid polymer.

4. The hydrogel composition of claim 1, wherein the composition comprises 0.5%-5% alginate polymer.

5. (canceled)

6. The hydrogel composition of claim 1, wherein the composition comprises 0.25%-5% hyaluronic acid polymer.

7. (canceled)

8. (canceled)

9. The hydrogel composition of claim 1, wherein the cell-instructive peptide comprises RGD (Arg-Gly-Asp).

10. The hydrogel composition of claim 1, wherein the cell-instructive peptide comprises YIGSR (Tyr-Ile-Gly-Ser-Arg).

11. The hydrogel composition of claim 1, wherein the cell-instructive peptide comprises IKVAV (Ile-Lys-Val-Ala-Val).

12. The hydrogel composition of claim 1, wherein the cell-instructive peptide comprises GFOGER (GGYGGGPC(GPP)5GFOGER(GPP)5GPC).

13. The hydrogel composition of claim 1, wherein the cell-instructive peptide comprises GHK (Gly-His-Lys).

14. The hydrogel composition of claim 1, wherein the cell-instructive peptide is an adhesion peptide.

15. The hydrogel composition of claim 1, further comprising a mineralized polymeric microsphere.

16. The hydrogel composition of claim 15, wherein the polymeric microsphere comprises polylactic acid (PLA), polyglycolide (PGA), poly(lactic-co-glycolic acid) (PLGA), or polycaprolactone (PCL).

17. The hydrogel composition of claim 15, wherein the polymeric microsphere comprises a coating.

18. The hydrogel composition of claim 17, wherein the coating comprises a ceramic, an apatite, or a bone-like mineral.

19. (canceled)

20. A method of producing the hydrogel composition of claim 1, the method comprising covalently coupling the cell-instructive peptide with hyaluronic acid and alginate, mixing the hyaluronic acid-cell-instructive peptide and alginate-cell-instructive peptide components together to form a solution, adding a polymeric microsphere to form a second solution, and gelling the second solution thereby forming the hydrogel composition.

21. (canceled)

22. (canceled)

23. (canceled)

24. (canceled)

25. (canceled)

26. The method of claim 20, wherein the cell-instructive peptide comprises RGD (Arg-Gly-Asp).

27. The method of claim 20, wherein the cell-instructive peptide comprises YIGSR (Tyr-Ile-Gly-Ser-Arg).

28. (canceled)

29. (canceled)

30. (canceled)

31. The method of claim 20, wherein the hydrogel composition further comprises a mineralized polymeric microsphere.

32. (canceled)

33. (canceled)

34. (canceled)

35. (canceled)

36. A method of administering the hydrogel composition of claim 1 to a subject, the method comprising administering the hydrogel composition to the subject.
